(19) United States
(12) Patent Application Publication
(10) Pub. No.: US 2025/0261677 A1
(43) Pub. Date: Aug. 21, 2025

(54) Confirming Diethylcarbamazine Levels using Near-Infrared Spectroscopy Detection in an Extruder

(71) Applicant: Rubicon Scientific, LLC, Sabetha, KS (US)

(72) Inventors: Bradley S. Strahm, Sabetha, KS (US); Cole M. Ahlvers, Topeka, KS (US); Stanley M. Teeter, Topeka, KS (US); Lon D. Lewis, Topeka, KS (US)

(73) Assignee: Rubicon Scientific, LLC, Sabetha, KS (US)

(21) Appl. No.: 19/055,633

(22) Filed: Feb. 18, 2025

Related U.S. Application Data
(60) Provisional application No. 63/555,439, filed on Feb. 20, 2024.

(51) Int. Cl. A23N 17/00; G01N 21/359; G01N 33/02

(52) U.S. Cl. CPC A23N 17/005; A23N 17/007; G01N 21/359; G01N 33/02

(57) ABSTRACT

A method is disclosed for producing an extruded daily ration feed product with a known concentration of diethylcarbamazine. The method involves mixing propylene glycol and diethylcarbamazine together to form an active blend with a predetermined ratio. The active blend and a feed mixture are then introduced into an extruder and forced through a die to form the extruded daily ration feed product. Inline near-infrared spectroscopy is used to detect the propylene glycol within the extruder. The detected level of propylene glycol is correlated to the concentration of diethylcarbamazine within the extruded daily ration feed product based on the predetermined ratio. This method provides a reliable and efficient way to ensure the desired concentration of diethylcarbamazine exists in the extruded daily ration feed product during the production process.

### CROSS REFERENCES

This U.S. Nonprovisional application claims the benefit of U.S. Provisional Application No. 63/555,439 entitled Confirming Diethylcarbamazine Levels using Near-Infrared Spectroscopy Detection in an Extruder, filed on 20 Feb. 2024, which is incorporated herein by reference in its entirety.

### REFERENCE TO RESEARCH

Not Applicable.

### REFERENCE TO CDS

Not Applicable.

### FIELD OF THE INVENTION

The present disclosure relates to a method for producing daily ration animal feed products for household pets that include a therapeutic amount of diethylcarbamazine based on detection of propylene glycol during an extrusion process.

### BACKGROUND

Animals, such as household pets, consume feed products daily. Extrusion has been employed as a rapid, continuous production process to prepare feed products, such as pet foods. Raw ingredients undergo mixing and preconditioning to ensure uniformity of a feed mixture fed into an extruder. While being extruded, the raw ingredients within the feed mixture are cooked by the high pressure, high shear, and high temperature environment created by the screws encased in the extruder barrel. The cooked product is pushed out through a small opening, called a die, to form and shape the feed mixture into an extruded feed product.

### SUMMARY

In some aspects, the techniques described relate to a method including the steps of: (a) mixing propylene glycol and diethylcarbamazine together to form an active blend having a known ratio of propylene glycol to diethylcarbamazine; (b) introducing the active blend and a feed mixture into an extruder; (c) forcing the feed mixture with the active blend through the die to form an extruded daily ration feed product; and (d) detecting the propylene glycol within the extruder with inline near-infrared spectroscopy, wherein the detected level of propylene glycol correlates to the concentration of diethylcarbamazine within the extruded daily ration feed product.

In some aspects, the techniques described relate to a method, further including the step of:
(a) tracking in real time any extruded daily ration feed product that is produced while the calculated concentration of diethylcarbamazine deviates from a desired concentration.

In some aspects, the techniques described relate to a method including the steps of: (a) mixing a carrier ingredient and an active ingredient together to form an active blend having a known ratio of carrier ingredient to active ingredient; (b) introducing the active blend and a feed mixture into an extruder; and (c) detecting the carrier ingredient within the extruder.

In some aspects, the techniques described relate to a method, further including the step of:
(a) calculating the concentration of the active ingredient based on an amount of carrier ingredient detected and the known ratio of carrier ingredient to active ingredient.

In some aspects, the techniques described relate to a method, further including the step of:
(a) increasing the amount of active blend introduced into the feed mixture if the calculated concentration of the active ingredient is below a desired concentration.

In some aspects, the techniques described relate to a method, further including the step of:
(a) decreasing the amount of active blend introduced into the feed mixture if the calculated concentration of the active ingredient is above a desired concentration.

In some aspects, the techniques described relate to a method, further including the step of:
(a) forcing the feed mixture through the die to form an extruded daily ration feed product.

In some aspects, the techniques described relate to a method, further including the step of:
(a) verifying, continuously, that the extruded daily ration feed product has a desired concentration of active ingredient in real time.

In some aspects, the techniques described relate to a method, further including the step of:
(a) tracking in real time any extruded daily ration feed product that is produced while the calculated concentration of the active ingredient deviates from a desired concentration.

In some aspects, the techniques described relate to a method, further including the step of:
(a) halting the extrusion process if the active ingredient deviates from the desired concentration within the feed mixture.

In some aspects, the techniques described relate to a method, further including the step of:
(a) transferring the extruded daily ration feed product away from downstream processing if the active ingredient deviates from the desired concentration.

In some aspects, the techniques described relate to a method, wherein the detected level of carrier ingredient correlates to the concentration of active ingredient within the extruded daily ration feed product.

In some aspects, the techniques described relate to a method, wherein the active ingredient does not have an established near-infrared absorption.

In some aspects, the techniques described relate to a method, wherein detecting is accomplished by inline near-infrared spectroscopy.

In some aspects, the techniques described relate to a method, wherein a near-infrared transmitter and a near-infrared detector are mounted to an extruder before a die of the extruder.

In some aspects, the techniques described relate to a method, further including the steps of:
(a) disposing a near-infrared transmitter on a first side of the extruder; and (b) disposing a near-infrared detector on a second side of the extruder, opposite the first side.

In some aspects, the techniques described relate to a method, wherein the carrier ingredient is detected through a cross-section of the feed mixture within a die spacer of the extruder.

In some aspects, the techniques described relate to a method, wherein the carrier ingredient is soluble in the feed mixture.

In some aspects, the techniques described relate to a method, wherein the carrier ingredient is propylene glycol.

In some aspects, the techniques described relate to a method, wherein the carrier ingredient is polyethylene glycol.

In some aspects, the techniques described relate to a method, wherein the carrier ingredient is approximately 1% of the feed mixture.

In some aspects, the techniques described relate to a method, wherein the carrier ingredient is introduced into the extruder at a concentration of between 5 grams per kilogram to 50 grams per kilogram.

In some aspects, the techniques described relate to a method, wherein the active ingredient is soluble in the carrier ingredient.

In some aspects, the techniques described relate to a method, wherein the active ingredient is diethylcarbamazine.

In some aspects, the techniques described relate to a method, wherein the active ingredient is introduced into the extruder at a concentration of between 200 milligrams per kilogram to 2,000 milligrams per kilogram.

The above advantages and features are of representative embodiments only, and are presented only to assist in understanding the invention. It should be understood that they are not to be considered limitations on the invention as defined by the claims. Additional features and advantages of embodiments of the invention will become apparent in the following description, from the drawings, and from the claims.

### BRIEF DESCRIPTION OF DRAWINGS

Aspects are illustrated by way of example, and not by way of limitation, in the accompanying drawings, wherein:

FIG. 1 depicts an example diagram of a feed production process for forming an extruded daily ration feed product containing a known amount of an active ingredient.

FIG. 2 depicts a general overview of a feed production system that might be used in the feed production process.

FIG. 3 depicts an extruder having an inline near-infrared spectroscopy transmitter and detector positioned at the die spacer.

FIG. 4 depicts a flowchart of an example method for confirming that an active ingredient is introduced into an extruded daily ration feed product at a controlled concentration.

### DETAILED DESCRIPTION

A method is disclosed for confirming that an active ingredient 133, such as diethylcarbamazine (DEC), is introduced into a feed product, such as an extruded daily ration feed product 135, at a controlled concentration. An active blend 132 is formed by mixing the active ingredient 133 with a carrier ingredient 134 at a predetermined ratio prior to an extrusion process. The active blend 132 may be introduced with a feed mixture 125 into an extruder 130 through active blend ports 232 disposed through a wall of the extruder 130. Inline near-infrared spectroscopy detects the carrier ingredient 134 within the feed mixture 125 to determine the concentration of the active ingredient 133 inside the extruder 130 during the extrusion process. The extruded daily ration feed product 135 is actively monitored, with immediate feedback, to track inconsistent concentrations of the active ingredient 133 during the extrusion process. Real time monitoring of concentrations of the active ingredient 133 during the extrusion process allows for timely adjustment in processing parameters. Desired concentrations of the active ingredient 133 are incorporated into the final feed product without relying on post-extrusion analysis techniques, such as laboratory testing which can be time-consuming and delay timely adjustments during the extrusion process.

The method provides a comprehensive solution allowing for accurate and efficient control during the production process 100 to obtain desired concentrations of an active ingredient 133 in the extruded daily ration feed product 135. Combination in steps of mixing the active blend 132, introducing the active blend 132 with the feed mixture 125 into the extruder 130, and detecting the concentration of the carrier ingredient 134, in real time, during the extrusion process helps alleviate current limitations in producing an extruded daily ration feed product 135 with a controlled concentration of an active ingredient 133. The method produces a daily ration feed product that may then be used in the feeding of domesticated household pets, such as cats and dogs.

The extruded daily ration feed product 135 produced by the extrusion process may contain respective quantities of protein, fat, and starch, together as part of the feed mixture 125, with the active blend 132. The active blend 132, including the active and carrier ingredients, are incorporated therein into the total feed mixture, including raw ingredients, meat 122, water 126, steam 124, and the dry blend 110, which are transformed into the cooked and formed feed product. The active ingredient 133 is incorporated at therapeutically effective amounts into the final feed product which becomes part of an animal's normal diet. A household pet consuming the feed product daily receives a maintenance quantity of the active ingredient 133, which is based on the animal's weight and dosage thresholds, so that therapeutic effects are realized without harmful side-effects.

Table 1, which follows, sets forth inclusion rates and ingredient proportions at different steps in a production process 100 of a first feed product:

TABLE 1

| Production Step | Ingredient | Addition Rate | Amount |
|---|---|---|---|
| EXTRUSION | Dry Blend | kg/hr. | 500.0 |
| | Emulsified Chicken | kg/hr. | 131.6 |
| | Salmon Oil | kg/hr. | 13.2 |
| | Active Blend | kg/hr. | 12.0 |
| ACTIVE BLEND | DEC Base | grams | 160.7 |
| | Propylene Glycol | kg | 3.42 |
| | Deionized Water | kg | 3.42 |
| | | Total (kg) | 7.17 |
| COATING | Dried Kibble | kg | 328.52 |
| | External Chicken Fat | kg | 21.4 |
| | AFB B18060 | kg | 7.1 |
| | | Total (kg) | 357.02 |
| CONFIRMATION | DEC in final product | mg/kg | 450 |
| | Propylene Glycol in | % | 0.96% |
| | final product | | |

Table 2, which follows, sets forth inclusion rates and ingredient proportions at different steps in a production process 100 of a second feed product:

TABLE 2

| Production Step | Ingredient | Addition Rate | Amount |
|---|---|---|---|
| EXTRUSION | Dry Blend | kg/hr. | 1500.0 |
| | Emulsified Chicken | kg/hr. | 394.7 |
| | Fish Oil | kg/hr. | 39.5 |
| | Active Blend | kg/hr. | 36.0 |
| ACTIVE BLEND | DEC Base | grams | 1036.4 |
| | Propylene Glycol | kg | 9.99 |
| | Deionized Water | kg | 9.99 |
| | | Total (kg) | 21.02 |
| COATING | Dried Kibble | kg | 988.58 |
| | External Chicken Fat | kg | 65.2 |
| | AFB B18060 | kg | 32.6 |
| | | Total (kg) | 1086.38 |
| CONFIRMATION | DEC in final product | mg/kg | 954 |
| | Propylene Glycol in | % | 0.9% |
| | final product | | |

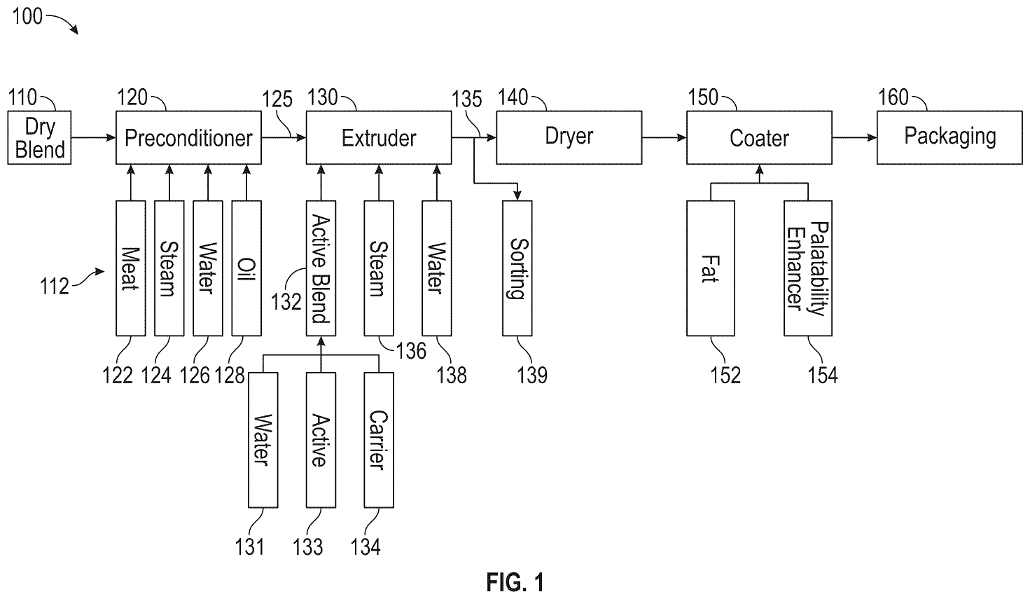

FIG. 1 depicts a diagram of a feed production process 100 that forms an extruded daily ration feed product 135 containing a known amount of an active ingredient 133. The continuous process may first involve providing an incoming stream of dry blend materials and an incoming stream of wet blend materials. The incoming streams of materials may be conditioned together, or separately, by a preconditioning system. Conditioning of the blend materials may be performed by preconditioning equipment such as a ribbon blender or preconditioner to ensure uniformity before blended materials enter the extruder 130 downstream of the preconditioning system.

As shown in FIG. 1, a preconditioner 120 may receive a dry blend 110 of granular, solid materials and a wet blend 112 of raw, moist materials. Alternatively, an extruder 130 may directly receive the dry blend 110 and wet blend 112. Preconditioner 120 may be used to pre-hydrate the blended materials and in some cases pre-cook the blended materials before feeding the blended materials into the extruder 130. Functions of the preconditioner 120 may include moisture adjustment and precooking of the blended materials prior to extrusion. During preconditioning, blended materials may be held in a warm, moist environment where they are mixed for a prescribed time, and then discharged into the preconditioner 120 or extruder 130. Preconditioning provides the benefits of improved product quality, reduced extruder wear, increased extruder capacity, and reduced power consumption.

The preconditioner 120 may be of a single-shaft design or a double-shaft design. The shaft, having mixing elements, may be rotated at relatively low or high speeds, resulting in retention times of 30 seconds or less, or 30 seconds or more, respectively. With the double-shaft design, the two shafts may have different dimensions, and rotate at different speeds, which results in improved mixing, and retention times of between 2 and 4 minutes. The preconditioner 120 can be operated at either atmospheric pressure or elevated pressure. Preconditioning at elevated pressure increases cooking temperatures above 100° C. to form steam, without providing an input of steam 124 which may be advantageous. During preconditioning, steam 124 and/or water 126 may be supplied to increase the temperature and moisture content of the blended materials. Steam 124 may be introduced from the bottom of the preconditioner 120 while water 126 that is hot (approximately 80-90° C.) may be added from the top through spray nozzles for uniform distribution. Alternatively, hot water 126 or liquid ingredients of the wet blend 112 may be introduced through liquid ports 220 disposed through a wall of the preconditioner 120.

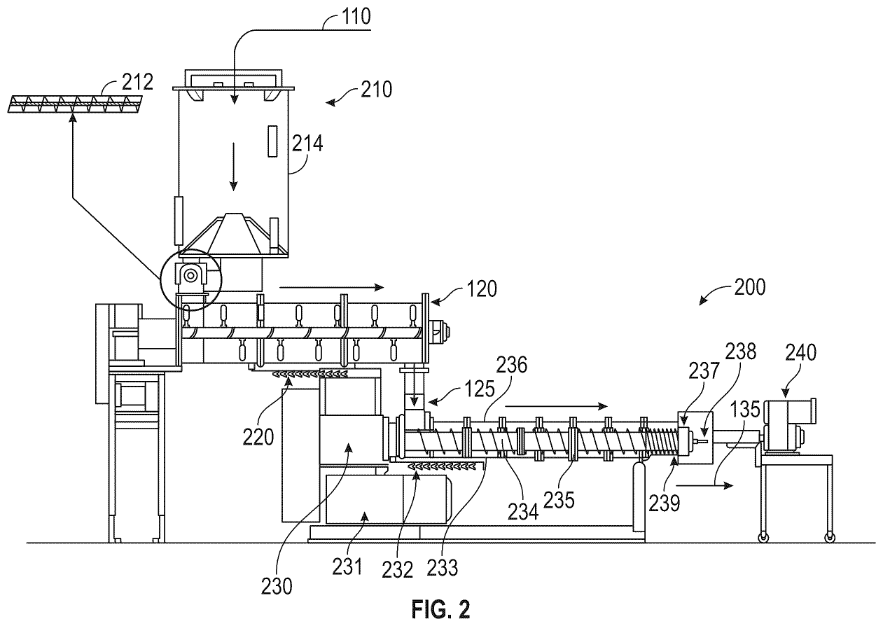

FIG. 2 shows a general overview of a feed production system 200 that might be used in the feed production process 100 depicted in FIG. 1. The preconditioner 120 may be mounted between a dry ingredient feeder 210, which may include a variable speed screw feeder 212 mounted to a bottom of a dry holding storage bin 214, and the extruder 130. The storage bin 214 may be used for the storage of the dry blend ingredients. The storage bin 214 provides a buffer of bulk material so that an extruder 130 has a continuous and stable supply of feed ingredients. The storage bin 214 may be equipped with a live bottom that has rotating blades to prevent bridging of the dry blend materials.

An incoming stream of dry blend materials may include dry farinaceous ingredients. Examples of ingredients for the dry blend 110 include flour derived from wheat, rice, and corn, corn gluten meal, poultry meal, brewer's yeast, sodium bicarbonate, ethoxyquin-O, potassium sorbate, sugar, glutamine peptide (GP), cheese powder, etc. The flow rate at which the incoming stream of dry blend material(s) is introduced into the preconditioner 120 or extruder 130 may be between 2 kilograms per hour (kg/hr.) and 2,000 kg/hr. The incoming flow rate of the dry blend material(s) may be further between 15 kg/hr. and 1,500 kg/hr., between 500 kg/hr. and 1,500 kg/hr., between 1,000 kg/hr. and 2,000 kg/hr., 200 kg/hr. and 800 kg/hr., between 20 kg/hr. and 200 kg/hr., between 30 kg/hr. and 180 kg/hr., between 40 kg/hr. and 160 kg/hr., between 50 kg/hr. and 140 kg/hr., between 60 kg/hr. and 120 kg/hr., between 70 kg/hr. and 100 kg/hr., or at approximately 80 kg/hr.

As shown in FIG. 1, the incoming stream of dry blend materials may be mixed with incoming stream(s) of wet blend materials. The incoming stream(s) of wet blend materials may include a blend of aqueous and non-aqueous materials, such as meat 122, including fresh, uncooked animal meat, emulsified meat such as emulsified chicken, oil 128 such as fish oil, water 126 such as deionized water, steam 124, etc. Wet blend materials may be introduced into the preconditioner 120 or directly into the extruder 130. In one example, the approximate flow rates at which the incoming stream of meat 122, steam 124, and oil 128 are introduced into the preconditioner 120 are 21.1 kg/hr. (26.3%), 19.2 kg/hr. (24%), and 35 g/min. (2.6%), respectively.

Optionally, prior to the step of preconditioning, the wet blend materials may be blended in a blender, with or without additives such as water, fat, tallow, syrup, nutraceuticals, and/or other additives such as grain- or legume-derived or dairy proteins. Wet ingredients may be metered either by volume with a volumetric meter or by mass with a mass flow meter. Liquid metering devices may include rotameters, differential pressure meters, fluid displacement meters, and velocity flow meters.

The flow rate at which the incoming stream of wet blend material(s) is introduced into the preconditioner 120 or extruder 130 may be between 1 kilogram per hour (kg/hr.) and 1,000 kg/hr. The incoming flow rate of the wet blend material(s) may be further between 50 kg/hr. and 500 kg/hr., between 100 kg/hr. and 200 kg/hr., between 300 kg/hr. and 500 kg/hr., between 2 kg/hr. and 90 kg/hr., between 4 kg/hr. and 80 kg/hr., between 6 kg/hr. and 70 kg/hr., between 8 kg/hr. and 50 kg/hr., between 10 kg/hr. and 40 kg/hr., or between 20 kg/hr. and 30 kg/hr.

Ingredient feeders used to feed the preconditioner 120 or extruder 130 may be selected from two types, a volumetric-type feeder, or a gravimetric-type feeder. Volumetric feeders deliver ingredients on a volume basis. A constant volume of the ingredients may be provided by the volumetric feeder but may not guarantee a constant mass flow rate due to changes in density of feed ingredients. Volumetric feeders of the single-screw kind provide a feed flow rate that is proportional to the screw speed. Use of a twin-screw improves feeding accuracy, but with higher manufacturing cost. Gravimetric feeders control feed flow rates based on the mass delivered. Therefore, a gravimetric feeder may be a more accurate feeding device to use in the feed production system 200. A gravimetric feeder monitors and meters the weight of ingredients gravimetrically and adjusts the feed rate accordingly, if any deviation is detected. Gravimetric metering may give more precision than volumetric metering. Alternatively, a weigh-belt or loss-in-weight feeder may be used in the feed production system 200 for gravimetric metering.

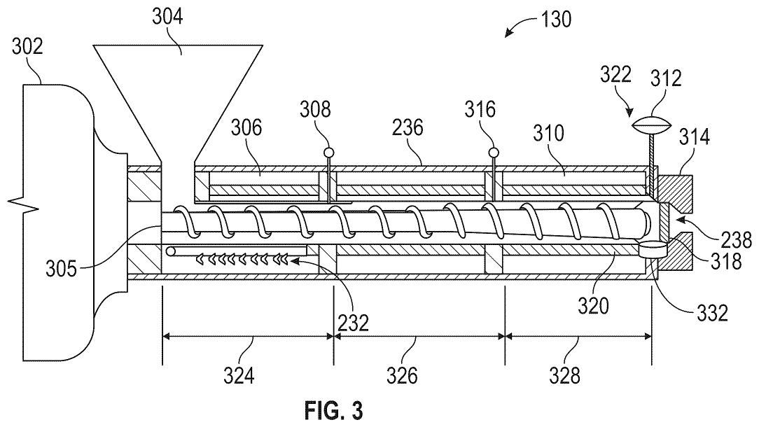

FIG. 3 shows that the extruder 130 may receive a feed mixture 125 from the preconditioner 120. The feed mixture 125 may be introduced through a feed hopper 304 into an inlet side or head end 233 of the extruder 130 adjacent a feed section 324. The extruder 130 may be of single-screw design, which may be easier to maintain. Twin-screw extruders may process the feed mixture 125 with a wider range of characteristics. Screws may have segments so that various screw configurations can be achieved to meet the processing requirements of the feed products. Various screw segments may have different processing functions, such as conveying, kneading, mixing, shearing, and compression, all of which may be configured independently or in combination to meet the processing requirements for the feed product.

As shown in FIG. 1, an active blend 132, which may be a liquid having a known ratio of carrier ingredient 134 to active ingredient 133, may be introduced into the extruder 130 with the feed mixture 125. The active blend 132 may be included in the feed product for the purpose of animal consumption, with the expected result of imparting and maintaining a therapeutic benefit in the animal at a dosage threshold that is specific to the animal's weight when consumed daily. The active blend 132 may be a substance that is not normally consumed as a food or not normally used as a characteristic ingredient of food.

Prior to the active blend 132 being introduced into the extruder 130, a known ratio of a carrier ingredient 134 to an active ingredient 133 may be mixed to form the active blend 132. The carrier ingredient 134 of the active blend 132 may be a substance that is used to dissolve, dilute, disperse, or otherwise modify the active ingredient 133 or a flavoring, food enzyme, nutrient, and/or therapeutic drug of the active blend 132. The carrier ingredient 134 may be added to the feed mixture 125 for nutritional, physiological, or other purposes of the feed product without altering its functioning, handling, application, or use. The carrier ingredient 134 may be soluble in the active blend 132. The carrier ingredient 134 may be soluble or insoluble in the feed mixture 125. The carrier ingredient 134 may be propylene glycol, polyethylene glycol, a lipid-based delivery system such as microemulsions, liposomes, solid lipid micro- or nanoparticles, a water-based delivery system, or include carriers derived from proteins or carbohydrates.

A carrier ingredient 134 used in the feed production process 100 may comprise propylene glycol. Propylene glycol may be blended with water 131 to form the carrier ingredient 134 of the active blend 132. An active ingredient 133 blended with propylene glycol and water 131 may be easier to detect with analytical methods using near-infrared spectroscopy. The active ingredient 133 may be soluble in the carrier ingredient 134. The carrier ingredient 134 may be soluble or insoluble in the other feed ingredients when mixed throughout the feed mixture 125. The carrier ingredient 134 may not be present in any other ingredients introduced into the feed mixture 125, so that the carrier ingredient 134 may be accurately detected as a component in the feed mixture 125. Detecting the level of the carrier ingredient 134 provides an accurate correlation so that the concentration of the active ingredient 133 can be factored based on the known ratio of carrier ingredient 134 to active ingredient 133 in the active blend 132.

In one example, the carrier ingredient 134 may be introduced into the extruder 130 at a concentration of between 5 grams per kilogram (g/kg) to 150 g/kg of the total weight of the feed mixture 125. The concentration at which the carrier ingredient 134 is introduced into the extruder 130 may be further between 50 g/kg to 150 g/kg, between 100 g/kg to 150 g/kg, between 75 g/kg to 125 g/kg, between 5 g/kg to 50 g/kg, between 5 g/kg to 45 g/kg, between 10 g/kg to 40 g/kg, between 20 g/kg to 50 g/kg, between 15 g/kg to 45 g/kg, between 20 g/kg to 40 g/kg, between 5 g/kg to 25 g/kg, between 5 g/kg to 25 g/kg, or between 5 g/kg to 15 g/kg of the total weight of the feed mixture 125.

In one example, the carrier ingredient 134 may be introduced into the extruder 130, with the feed mixture 125, at a processing rate of approximately 1% (percentages based on weight per time) of the total feed mixture processed during the feed production extrusion process. A processing rate of approximately 1% inclusion of the carrier ingredient 134 may be targeted so that after topical ingredients are added the carrier ingredient 134 becomes diluted to less than 1% of the extruded feed product. In this case, the carrier ingredient 134 may not have to be listed on a package label. Processing rates of inclusion of the carrier ingredient 134 into the total feed mixture may be between about 0.5% to 5%, or further between about 1% to 5%, between about 1% to 3%, between about 2% to 4%, between about 3% to 5%, between about 0.5% to 1.5%, between about 0.7% to 1.3%, between about 0.6% to 1.0%, or between about 0.9% to 1.1% of the total feed mixture during the feed production extrusion process.

The active ingredient 133 of the active blend 132 may be a substance such as a food enzyme, nutrient, pharmaceutical, nutraceutical, antibiotic, steroid, analgesic, angiotensin-converting enzyme (ACE) inhibitor, anti-inflammatory agent, endoectacide (e.g. dewormer such as heartworm preventative drug), ectoparasiticide (e.g. drug effective against fleas and ticks), and/or other therapeutic drug selected from a wide range of drugs that is used to treat, maintain, prevent, or otherwise furnish pharmacological activity in the animal that consumes the active ingredient 133. The active blend 132 may be comprised of multiple active ingredients. An active ingredient 133 may be added to the feed mixture 125 for nutritional, physiological, or other purposes of the feed product without altering its functioning, handling, application, or use. The active ingredient 133 may be soluble in the carrier ingredient 134 or the water 131 of the active blend 132.

The active ingredient 133 may be one, or any appropriate combination, of the following ingredients such as Methoprene (insect growth regulator), Lufenuron (insect growth regulator, chemically dissimilar to Methoprene), Praziquantel (tapeworm treatment), Enrofloxacin (potent broad spectrum antibiotic), Dexamethasone (steroid of the cortisone type), Ibuprofen (non-steroidal anti-inflammatory drug) Fenbendazole (mammal dewormer), Oxytetracycline (widely used antibiotic), Ivermectin/Methoprene/Praziquantal cocktail (antiparasitical combination), Imidaccopria (imidacloprid), Amoxicillin (broad spectrum antibiotic), Tribrissen (antibiotic), Doramectin (broad spectrum dewormer and anthelmintic), Selamectin, Moxidectin, Milbemycin Oxime, Eprinomectin, or a broader range of drugs than listed here.

An active ingredient 133 that may be used in the feed production process 100 is diethylcarbamazine. For an active ingredient 133, such as diethylcarbamazine which has a weaker near-infrared spectroscopy signature, the active ingredient 133 may be mixed with the carrier ingredient 134. For an active ingredient 133 having a stronger near-infrared spectroscopy signature, the active ingredient 133 may be measured directly, without a carrier ingredient 134, within the extruder 130 along an extruder barrel assembly 236 or at a die spacer 237.

In one example, the concentration at which the active ingredient 133 is introduced into the extruder 130 may be at a concentration of between 2 milligrams per kilogram (mg/kg) to 2,000 mg/kg of the total weight of the feed mixture 125. The concentration at which the active ingredient 133 is introduced into the extruder 130 may be further between 20 mg/kg to 1,500 mg/kg, between 200 mg/kg to 2,000 mg/kg, between 100 mg/kg to 1,000 mg/kg, between 200 mg/kg to 1,000 mg/kg, between 500 mg/kg to 1,500 mg/kg, between 700 mg/kg to 1,100 mg/kg, between 800 mg/kg to 1,100 mg/kg, between 400 mg/kg to 800 mg/kg, or between 500 mg/kg to 750 mg/kg of the total weight of the feed mixture 125.

In one example, the active ingredient 133 may be introduced into the extruder 130, with the feed mixture 125, at a processing rate of approximately 0.1% (percentages based on weight per time) of the total feed mixture processed during the feed production extrusion process. A processing rate of 0.1% inclusion may be targeted so that after topical ingredients are added the active ingredient 133 becomes diluted to less than 0.1% of the extruded feed product. Processing rates of the active ingredient 133 with inclusion into the total feed mixture may be between 0.0002% to 0.2%, or further between 0.004% to 0.2%, between 0.0008% to 0.2%, between 0.001% to 0.2%, between 0.002% to 0.15%, between 0.02% to 0.2%, between 0.01% to 0.1%, between 0.02% to 0.1%, between 0.05% to 0.15%, between 0.07% to 0.11%, between 0.08% to 0.11%, or between 0.04% to 0.08% of the total feed mixture during the feed production extrusion process.

An automated mixing system may be used to mix the active blend 132 and the feed mixture 125 prior to being introduced, together, through a wall of the extruder 130, such as through active blend ports 232 disposed in a feed section 324 of the extruder 130. Alternatively, the automated mixing system may introduce the active blend 132 and the feed mixture 125, separately on an individual basis, into the extruder 130. The automated mixing system may use pumps and valves to control the flow rates of the active blend 132 and the feed mixture 125 into the extruder 130. The automated system may improve the accuracy and consistency of the mixing process.

The active ingredient 133 may be pre-mixed throughout the carrier ingredient 134 at a known ratio prior to being introduced into the extruder 130. The ratio may be based on concentration. The ratio of the carrier ingredient 134 to active ingredient 133 incorporated into the feed mixture 125 may be between a range of about 20,000:1 to 2:1. The ratio of carrier ingredient 134 to active ingredient 133 may be further between a range of 500:1 and 5:1, between 400:1 and 4:1, between 300:1 and 3:1, between 200:1 and 2:1, between 100:1 and 2:1, between 100:1 and 10:1, between 200:1 and 20:1, between 300:1 and 30:1, between 400:1 and 40:1, or between 500:1 and 50:1.

The active ingredient 133 may be pre-mixed throughout the carrier ingredient 134 at known concentrations prior to being introduced into extruder 130. The concentration of active ingredient 133 to carrier ingredient 134 that is to be incorporated into the feed mixture 125 may be between about 0.4 g of active ingredient per 1 kg of carrier ingredient to about 400 g of active ingredient per 1 kg of carrier ingredient. The concentration of active ingredient to carrier ingredient may be further between about 0.8 g of active ingredient per 1 kg of carrier ingredient to about 80 g of active ingredient per 1 kg of carrier ingredient, between about 2 g of active ingredient per 1 kg of carrier ingredient to about 200 g of active ingredient per 1 kg of carrier ingredient, between about 20 g of active ingredient per 1 kg of carrier ingredient to about 200 g of active ingredient per 1 kg of carrier ingredient, between about 4 g of active ingredient per 1 kg of carrier ingredient to about 400 g of active ingredient per 1 kg of carrier ingredient, between about 40 g of active ingredient per 1 kg of carrier ingredient to about 400 g of active ingredient per 1 kg of carrier ingredient, between about 40 g of active ingredient per 1 kg of carrier ingredient to about 100 g of active ingredient per 1 kg of carrier ingredient, or any range between any combination of these concentrations.

The flow rate at which the active blend 132 is introduced into the extruder 130 may be between 0.01 kilograms per hour (kg/hr.) and 100 kg/hr. The incoming flow rate of the active blend 132 into the extruder 130 may be further between 10 kg/hr. and 80 kg/hr., between 10 kg/hr. and 60 kg/hr., between 10 kg/hr. and 40 kg/hr. between 0.2 kg/hr. and 10 kg/hr., between 0.4 kg/hr. and 8 kg/hr., between 0.6 kg/hr. and 6 kg/hr., between 0.8 kg/hr. and 4 kg/hr., between 1 kg/hr. and 2 kg/hr., or at approximately 2 kg/hr.

In one example, the active blend 132 is introduced into the feed mixture 125 in the extruder 130 at a processing rate of approximately 2.4% (percentages based on weight per time) of the total feed mixture during the feed production extrusion process. Processing rates of the active blend 132 in the total feed mixture may be between about 1% to 10%, or further between about 4% to 8%, between about 2% to 6%, between about 3% to 6%, between about 1% to 5%, between about 2% to 5%, between about 3% to 5%, between about 1% to 3%, between about 2% to 4%, or between about 2% to 3% of the total feed mixture during the feed production extrusion process.

In one example, the active blend 132 is introduced into the feed mixture 125 in the extruder 130 at a flow rate of approximately 32 grams per min (g/min.). The flow rate at which the active blend 132 is introduced into the feed mixture 125 in the extruder 130 may be between 2 g/min. and 200 g/min. The incoming flow rate of the active blend 132 may be further between 15 g/min. and 150 g/min., between 50 g/min. and 150 g/min., between 100 g/min. and 200 g/min., 20 g/min. and 80 g/min., between 2 g/min. and 20 g/min., between 30 g/min. and 180 g/min., between 40 g/min. and 160 g/min., between 50 g/min. and 140 g/min., between 60 g/min. and 120 g/min., between 70 g/min. and 100 g/min., between 10 g/min. and 100 g/min., between 20 g/min. and 40 g/min., or at approximately 30 g/min.

The active ingredient 133, which is incorporated into the carrier ingredient 134 to form the active blend 132, mixes throughout the feed mixture 125 within the extruder 130 during the extrusion process. The carrier ingredient 134 may be detectable during the extrusion process. An active ingredient 133, such as diethylcarbamazine, may be incorporated into a carrier ingredient 134, such as a blend of propylene glycol and water 131. Analytical methods such as near-infrared spectroscopy may allow the carrier ingredient 134 mixed throughout the feed mixture 125 to be detected so that the amount of active ingredient 133 may be calculated during the extrusion process.

The carrier ingredient 134 may be detectable within an extruder barrel 320 of the barrel assembly 236 that has a hardened liner. The barrel 320 of the extruder 130 encases the screw assembly 305 that may comprise a screw 234 or a set of screws having root diameters that increase, decrease, or a combination of both. Often, the barrel 320 is surrounded or jacketed for heating by steam 136 within a steam jacket 310 and for cooling by water 138 within a water jacket 306. In one example, the approximate flow rate at which the incoming stream of steam 136 is introduced into the extruder 130 is 8 kg/hr. (10%). The heating can also be accomplished by mounting electrical heating units on the barrel 320. The inner surface of the barrel 320 may be smooth, grooved, or fluted. Different shearing conditions and pressure profiles may be suited to produce the feed product based on screw configurations. A plurality of sensors that measure pressure and/or temperature, such as thermocouples, may be mounted within a plurality of ports 308, 316 disposed along functional sections between intake and discharge ends of the barrel 320 to monitor the feed mixture 125.

The extruder barrel assembly 236 may have two or more functional sections. As shown in FIG. 3, three functional sections of the barrel 320 include a feed section 324, a compression section 326, and a metering section 328. A continuous flow of mass forms within the barrel 320 in the feed section 324. The feed section 324 pushes the feed mixture 125 that is incoming towards the metering section 328. Feed mixture 125 conveyed to the metering section 328 may melt due to input of thermal energy through the wall of the extruder barrel 320. Feed mixture 125 may be heated by an output of thermal energy from the mechanical energy exerted upon the screw assembly 305 by a drive gear reducer and thrust bearing 302 powered by a main drive motor 231 which may be enclosed within a main drive housing 230. Additional mixing and melting of the feed mixture 125 may occur within the compression section 326. The feed mixture 125 may be fully mixed and cooked before exiting the metering section 328 at a die assembly 238.

The extruder barrel assembly 236 may have various injection ports, such as active blend ports 232 for injection of the active blend 132, additional feeding ports for the feed mixture 125, and monitoring ports for sensors that are disposed through the barrel wall along a longitudinal length of the barrel 320. Monitoring assemblies may be stationed between shear locks 235 of any of the extruder sections and/or outside extruder sections, such as between the terminal end 239 of the screw assembly 305 and the die assembly 238. In one example, a monitoring assembly 322 may be positioned prior to the die assembly 238 to monitor the feed mixture 125 within one of the extruder sections. In another example, a monitoring assembly 322 may be positioned after the screw assembly 305 to monitor the feed mixture 125 within the die assembly 238. The die assembly 238 may incorporate a die spacer 237. The die spacer 237 defines a space downstream of the metering section 328 that is between the terminal end 239 of the screw assembly 305 in the extruder barrel 320 and the upstream side of a breaker plate 318 of the die assembly 238.

A monitoring assembly 322 may be disposed through the extruder barrel 320 to detect the level of carrier ingredient 134 therein. The carrier ingredient 134 in the feed mixture 125 is detectable within the extruder 130 during the extrusion process. The purpose of detecting the level of carrier ingredient 134 is to confirm that the level of active ingredient 133, which is correlated to the level of carrier ingredient 134 based on a known ratio, is at the desired level of active ingredient 133 intended for the final feed product. The monitoring assembly 322 may be disposed at the terminal end 239 of the metering section 328 to monitor feed mixture 125 within the die spacer 237. The feed mixture 125 within the extruder 130 may be monitored with remote sensing by the monitoring assembly 322. Remote sensing may include the acquisition of visible, near infrared, and short-wave infrared images in several broad wavelength bands.

For example, whereas the active ingredient 133 may be undetectable by near-infrared spectroscopy, or insubstantially detectable at low dosage levels, the carrier ingredient 134 may be detectable by near-infrared spectroscopy. Near-infrared spectroscopy is a form of analysis using the near-infrared region of the electromagnetic spectrum called near-infrared (NIR) light. NIR spectroscopy may be used to identify and quantify different materials in the feed mixture 125. This method may apply this specific type of analysis during the extrusion process.

As shown in FIG. 3, the monitoring assembly 322 includes a NIR transmitter 312 and a NIR detector 332. The NIR transmitter 312 and the NIR detector 332 are mounted inline on opposing sides of the die spacer 237. The NIR transmitter 312 and the NIR detector 332 may be disposed downstream of the extruder barrel 320 and before a die 314 of the die assembly 238. In one example, the NIR transmitter 312 and the NIR detector 332 are disposed within the die spacer 237 and directly downstream of the terminal end 239 of the screw 234 and upstream of the breaker plate 318. The NIR transmitter 312 is shown mounted on a first sidewall of the extruder 130. The NIR transmitter 312 is structured to transmit NIR light through the space disposed between the terminal end 239 of the screw assembly 305 and the interior side of the die breaker plate 318. The NIR detector 332 is mounted on a second sidewall of the extruder 130 facing the first sidewall that is opposite. The NIR detector 332 is structured to detect any unabsorbed NIR light being transmitted through the feed mixture 125 in the space within the die spacer 237. Alternatively, the NIR transmitter 312 and the NIR detector 332 may be mounted on the same side of the extruder 130 in a case where unabsorbed NIR light is reflected by the feed mixture 125 or a reflector on the opposing side.

Measuring the amount of NIR light scattered, reflected, absorbed, or passed through the feed mixture 125 during the extrusion process, in real time, assists in determining the feed mixture's properties, such as identifying and quantifying the feed ingredients, without having to conduct post-extrusion process analyses. For example, an absorption curve may be established for different levels of carrier ingredient 134 in the feed mixture 125 based on transmission of unabsorbed NIR light to the NIR detector 332. The difference between the amount of transmitted NIR light and the amount of detected NIR light gives the amount of absorbed NIR light. The level of absorbed NIR light may be associated with the level of carrier ingredient 134 within the feed mixture 125. Based on the level of carrier ingredient 134 detected, the level of active ingredient 133 may be determined based on the known ratio of carrier ingredient 134 to active ingredient 133 which is pre-determined at formation of the active blend 132.

The NIR light passes through a cross-section of the feed mixture 125 within the extruder 130 between the NIR transmitter 312 and the NIR detector 332 for detection of the carrier ingredient 134. The detected level of carrier ingredient 134 correlates to the concentration of the active ingredient 133 within the feed mixture 125 as it is formed into the extruded daily ration feed product 135. The amount of absorbed NIR light by the carrier ingredient 134 may be detected within the die spacer 237 and used to determine the concentration of active ingredient 133 that has been incorporated into the feed mixture 125 during the extrusion process. With more absorption of NIR light by the carrier ingredient 134, the more active ingredient 133 can be inferred to be within the feed mixture 125. With less absorption of NIR light by the carrier ingredient 134, the less active ingredient 133 can be inferred to be within the feed mixture 125.

The concentration of active ingredient 133, which may be undetectable during the extrusion process, may be calculated based on a detectable amount of the carrier ingredient 134 within the feed mixture 125. Inferring the concentration of the active ingredient 133 from the calculation, which is based on the known ratio of carrier ingredient 134 to active ingredient 133, allows real time tracking of the final feed product during the production process. Correlation between the carrier ingredient 134 and the active ingredient 133 may be based on another parameter. Other parameters, such as weight, volume, density, or pressure readings, may be detectable and utilized in the carrier ingredient 134 detection method while the active blend 132 is incorporated into the feed mixture 125 within the extruder 130. Preset thresholds may be established for the level of carrier ingredient 134 detected to verify if deviations in the concentration of the active ingredient 133 are occurring during the production process.

Verification that a desired concentration of active ingredient 133 is being incorporated into the feed mixture 125 may occur continuously, in real time, during the production process. If the calculated concentration of the active ingredient 133 is below a desired concentration, then the amount of active blend 132 being introduced into the feed mixture 125 may be increased. If the calculated concentration of the active ingredient 133 is above a desired concentration, then the amount of active blend 132 being introduced into the feed mixture 125 may be decreased.

The feed mixture 125, incorporating the desired amount of active ingredient 133, may be forced through a die assembly 238 of the extruder 130 to form the extruded daily ration feed product 135. The die assembly 238 may be mounted on the outlet end of the barrel 320. The die assembly 238 may hold the feed mixture 125 within the extruder barrel 320. Holding the feed mixture 125 within the extruder barrel 320 may provide time for the screw assembly 305 to impart shear energy into the feed mixture 125. Holding the feed mixture 125 in the barrel 320 may also provide time for the monitoring assembly 322 to detect the carrier ingredient 134 through a cross section of the feed mixture 125.

The die assembly 238 of the extruder 130 may include the die breaker plate 318. The die breaker plate 318 is mounted at the outlet end of the barrel 320. The die breaker plate 318 restricts and forms the feed mixture 125 into an extruded daily ration feed product 135 during the production process. Adjusting the die 314 opening, controls the pressure, retention time, and the dimensions and shape of the final feed product. The die assembly 238 may have one or more openings where the feed mixture 125 is forced through the die plate 314 to form the extruded daily ration feed product 135.

The extruded daily ration feed product 135 may be cut into finite lengths by a cutter 240, such as a variable speed knife. The cutter 240 may be a group of rotating knives mounted to the die plate 314. The rotational speed of the knives may be variable or continuous. The length of the extruded pieces may be determined by the number of knives mounted on the die plate 314 and the rotational speed of the knives.

As shown in FIG. 1, the extruded daily ration feed product 135 may transfer to a dryer 140 for drying of the extrudate. Subsequently, the extruded daily ration feed product 135 may transfer to a coater 150 for coating of the extrudate with a palatability enhancer 154 that may or may not be blended with fat 152, such as chicken fat. Alternatively, the extruded daily ration feed product 135 may transfer to a sorting apparatus 139 for sorting and bypass of the extrudate from further downstream processing.

The active ingredient 133 may be present in the extruded daily ration feed product 135 at a level of about 2 mg/kg of the feed product, further from about 20 mg/kg to about 10,000 mg/kg of the feed product, from about 200 mg/kg to about 2,000 mg/kg of the feed product, and from about 200 mg/kg to about 1,000 mg/kg of the feed product.

Tracking the calculated concentration of active ingredient 133 during the extrusion process also provides real time identification of any deviations from a desired concentration of active ingredient 133 in the extruded daily ration feed product 135. If the calculated concentration of active ingredient 133 processed does not correlate to the desired concentration of active ingredient 133 intended for the extruded daily ration feed product 135 produced, then the production process 100 may be discontinued. Alternatively, the extruded daily ration feed product 135 produced with a calculated concentration of active ingredient 133 that deviates from the desired concentration may be automatically tracked and sorted into a segregation bin, all without halting the production process. If the calculated concentration of active ingredient 133 processed does correlate to the desired concentration of active ingredient 133 intended for the extruded daily ration feed product 135 produced, then the production process 100 may be continued.

In one mode, where high volumes of feed product may be processed, a sorting apparatus 139 may be employed in the feed production system 200. The sorting apparatus 139 may be disposed at a discharge end of the extruder 130 or downstream of the extruder 130. Any extruded feed product that has an amount of carrier ingredient 134 that deviates from an acceptable established criteria may be sorted by the sorting apparatus 139. The sorting apparatus 139 may transfer extruded feed product having deviations with a sorting apparatus 139 in order to bypass downstream processing. The extruded feed product that bypasses downstream processing may be utilized in alternative products, corrected, or discarded. Once the detection method senses a correct amount of carrier ingredient 134, the extruded feed product may be sent to downstream processing and final packaging 160.

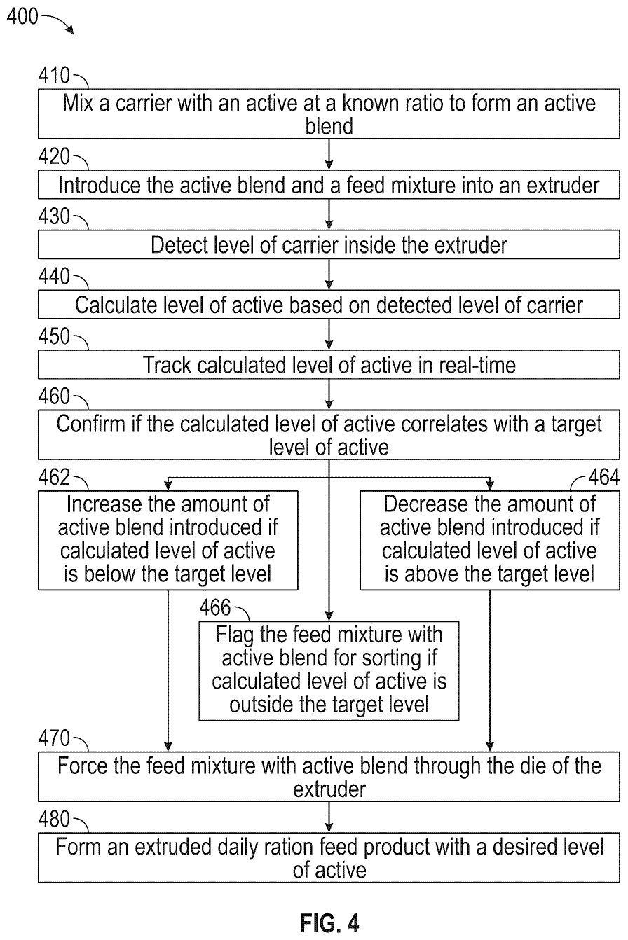

FIG. 4 is a flowchart of an example method 400 for confirming that an active is introduced into an extruded daily ration feed product at a controlled concentration. An active is mixed with a carrier to form an active blend with a known ratio of carrier to active at step 410. The active blend is introduced with a feed mixture into an extruder at step 420. The level of carrier is detected inside the extruder at step 430. The level of carrier may be detected within the extruder with inline near-infrared spectroscopy. The detected level of carrier may correlate to the level of active. The level of active is calculated based on the detected level of carrier at step 440. The calculated level of active is tracked in real time during the extrusion process at step 450. The calculated level of active is confirmed, whether it correlates with a target level of active at step 460. Upon completion of step 460, one of three substeps will occur. At substep 462, the amount of active blend introduced into the extruder will be increased if the calculated level of active is below the target level. At substep 464, the amount of active blend introduced into the extruder will be decreased if the calculated level of active is above the target level. At substep 466, the feed mixture with active blend is flagged for sorting by a sorting apparatus if the calculated level of active is outside a desired range of the target level. The feed mixture with the active blend is forced through the die of the extruder to form an extruded daily ration feed product at step 470. The extruded daily ration feed product is formed with a desired level of active in real time at step 480. After the die, the extruded daily ration feed product may be packaged with confirmation of the level of active present within the feed product, without any outside analysis.

It is understood that the invention is not confined to the particular construction and arrangement of parts herein described. That although the drawings and specification set forth a preferred embodiment, and although specific terms are employed, they are used in a description sense only and embody all such forms as come within the scope of the following claims.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims.

For the convenience of the reader, the above description has focused on a representative sample of all possible embodiments, a sample that teaches the principles of the invention and conveys the best mode contemplated for carrying it out. Throughout this application and its associated file history, when the term “invention” is used, it refers to the entire collection of ideas and principles described; in contrast, the formal definition of the exclusive protected property right is set forth in the claims, which exclusively control. The description has not attempted to exhaustively enumerate all possible variations. Other undescribed variations or modifications may be possible. Where multiple alternative embodiments are described, in many cases it will be possible to combine elements of different embodiments, or to combine elements of the embodiments described here with other modifications or variations that are not expressly described. A list of items does not imply that any or all of the items are mutually exclusive, nor that any or all of the items are comprehensive of any category, unless expressly specified otherwise. In many cases, one feature or group of features may be used separately from the entire apparatus or methods described. Many of those undescribed variations, modifications and variations are within the literal scope of the following claims, and others are equivalent.

We claim:

1. A method comprising the steps of:
a. mixing propylene glycol and diethylcarbamazine together to form an active blend having a known ratio of the propylene glycol to the diethylcarbamazine;
b. introducing the active blend and a feed mixture into an extruder;
c. forcing the feed mixture with the active blend through a die of the extruder to form an extruded daily ration feed product; and
d. detecting the propylene glycol within the extruder with inline near-infrared spectroscopy, wherein a detected level of the propylene glycol correlates to a concentration of the diethylcarbamazine within the extruded daily ration feed product.

2. The method of claim 1, further comprising the step of:
a. tracking in real time any extruded daily ration feed product that is produced while a calculated concentration of the diethylcarbamazine deviates from a desired concentration.

3. A method comprising the steps of:
a. mixing a carrier ingredient and an active ingredient together to form an active blend having a known ratio of the carrier ingredient to the active ingredient;
b. introducing the active blend and a feed mixture into an extruder; and
c. detecting the carrier ingredient within the extruder.

4. The method of claim 3, further comprising the step of:
a. calculating a concentration of the active ingredient based on an amount of the carrier ingredient detected and the known ratio of the carrier ingredient to the active ingredient.

5. The method of claim 4, further comprising the step of:
a. increasing the amount of the active blend introduced into the feed mixture if a calculated concentration of the active ingredient is below a desired concentration.

6. The method of claim 4, further comprising the step of:
a. decreasing the amount of the active blend introduced into the feed mixture if a calculated concentration of the active ingredient is above a desired concentration.

7. The method of claim 4, further comprising the step of:
a. forcing the feed mixture through a die of the extruder to form an extruded daily ration feed product.

8. The method of claim 7, further comprising the step of:
a. verifying, continuously, that the extruded daily ration feed product has a desired concentration of the active ingredient in real time.

9. The method of claim 7, further comprising the step of:
a. tracking in real time any extruded daily ration feed product that is produced while the calculated concentration of the active ingredient deviates from a desired concentration.

10. The method of claim 9, further comprising the step of:
a. transferring the extruded daily ration feed product to a sorting apparatus if the calculated concentration of the active ingredient deviates from the desired concentration.

11. The method of claim 7, wherein a detected level of the carrier ingredient correlates to the concentration of the active ingredient within the extruded daily ration feed product.

12. The method of claim 3, wherein the active ingredient does not have an established near-infrared absorption.

13. The method of claim 3, wherein the step of detecting is accomplished by inline near-infrared spectroscopy.

14. The method of claim 3, wherein a near-infrared transmitter and a near-infrared detector are mounted to the extruder before a die of the extruder.

15. The method of claim 3, further comprising the steps of:
a. disposing a near-infrared transmitter on a first side of the extruder; and
b. disposing a near-infrared detector on a second side of the extruder, opposite the first side.

16. The method of claim 3, wherein the carrier ingredient is detected through a cross-section of the feed mixture within a die spacer of the extruder.

17. The method of claim 3, wherein the carrier ingredient is soluble in the feed mixture.

18. The method of claim 3, wherein the carrier ingredient is propylene glycol.

19. The method of claim 3, wherein the carrier ingredient is polyethylene glycol.

20. The method of claim 3, wherein the carrier ingredient is approximately 1% of the feed mixture.

21. The method of claim 3, wherein the carrier ingredient is introduced into the extruder at a concentration of between 5 grams per kilogram to 50 grams per kilogram of the feed mixture.

22. The method of claim 3, wherein the active ingredient is soluble in the carrier ingredient.

23. The method of claim 3, wherein the active ingredient is diethylcarbamazine.

24. The method of claim 3, wherein the active ingredient is introduced into the extruder at a concentration of between 200 milligrams per kilogram to 2,000 milligrams per kilogram of the feed mixture.
